SEQUENCE LISTING (1 sequence)

SEQ ID NO: 1 - DNA, 28 bp, synthetic construct
  source 1..28: mol_type = other DNA
aaaaaaaaaatgtaaaacgacggccagt

(19) United States
(12) Patent Application Publication
(10) Pub. No.: US 2024/0254532 A1
(43) Pub. Date: Aug. 1, 2024

(54) INITIATOR PARTICLE FOR DNA SYNTHESIS

(71) Applicant: Lenovo (United States) Inc., Morrisville, NC (US)

(72) Inventors: Henrique da Costa Oliveira, Sao Paulo (BR); Joâo Lucas Maehara Said Dos Reis, Sao Paulo (BR); Joãko Henrique Diniz Brandao Gervasio, Sao Paulo (BR); João Bosco Pesquero, Sao Paulo (BR); Natalia Neto Pereira Cerize, Sao Paulo (BR)

(21) Appl. No.: 18/162,429

(22) Filed: Jan. 31, 2023

(51) Int. Cl. C12P 19/34; B01J 19/00; C12N 9/12

(52) U.S. Cl. CPC C12P 19/34; B01J 19/0046; C12N 9/1264; C12Y 207/07031

(57) ABSTRACT

According to various aspects of the present disclosure, an initiator particle for enzymatic DNA synthesis includes a substrate. The substrate can include silver, gold, glass, iron, or a combination thereof. The initiator particle also includes a linker molecule functionalized to the substrate. The initiator particle also includes an initiator oligonucleotide sequence functionalized to the linker molecule.

### BACKGROUND

Nucleic acid digital data storage is a robust approach to encoding and storing information over long periods of time where data is stored at higher densities than magnetic tape or hard drive storage systems.

### INCORPORATION BY REFERENCE OF SEQUENCE LISTING

A Sequence Listing is provided herewith as an xml file, “2341683.xml” created on Jun. 8, 2023, and having a size of 1,767 bytes. The content of the xml file is incorporated by reference herein in its entirety.

### SUMMARY OF THE INVENTION

Various aspects disclosed relate to an initiator particle for enzymatic DNA synthesis that includes a substrate. The substrate can include silver, gold, glass, iron, or a combination thereof. The initiator particle also includes a linker molecule functionalized to the substrate. The initiator particle also includes an initiator oligonucleotide sequence functionalized to the linker molecule.

Various aspects disclosed relate to an initiator particle for enzymatic DNA synthesis. The initiator particle includes a substrate. The substrate can include silver, gold, glass, iron, or a combination thereof. The initiator particle also includes a linker molecule functionalized to the substrate. The initiator particle also includes an initiator oligonucleotide sequence functionalized to the linker molecule. The initiator particle further includes a coded oligomer sequence bonded to the initiator oligonucleotide.

Various aspects disclosed relate to a method of making an initiator particle for enzymatic DNA synthesis. The initiator particle for enzymatic DNA synthesis includes a substrate. The substrate can include silver, gold, glass, iron, or a combination thereof. The initiator particle also includes a linker molecule functionalized to the substrate. The initiator particle also includes an initiator oligonucleotide sequence functionalized to the linker molecule.

Various aspects disclosed relate to a method of synthesizing a single stranded DNA oligomer. The method includes contacting an initiator particle with a nucleotide and an enzyme. The initiator particle for enzymatic DNA synthesis includes a substrate. The substrate can include silver, gold, glass, iron, or a combination thereof. The initiator particle also includes a linker molecule functionalized to the substrate. The initiator particle also includes an initiator oligonucleotide sequence functionalized to the linker molecule. The single stranded DNA oligomer is built from the 3′ end of the initiator oligonucleotide sequence.

Various aspects disclosed relate to a DNA synthesis device. The DNA synthesis device includes a reaction chamber. An initiator particle for enzymatic DNA synthesis is located at least partially within the reaction chamber. The initiator particle for enzymatic DNA synthesis includes a substrate. The substrate can include silver, gold, glass, iron, or a combination thereof. The initiator particle also includes a linker molecule functionalized to the substrate. The initiator particle also includes an initiator oligonucleotide sequence functionalized to the linker molecule. The single stranded DNA oligomer is built from the 3′ end of the initiator oligonucleotide sequence.

Various aspects disclosed relate to an information storage system. The system includes a DNA synthesis device. The DNA synthesis device includes a reaction chamber. An initiator particle for enzymatic DNA synthesis is located at least partially within the reaction chamber. The initiator particle for enzymatic DNA synthesis includes a substrate. The substrate can include silver, gold, glass, iron, or a combination thereof. The initiator particle also includes a linker molecule functionalized to the substrate. The initiator particle also includes an initiator oligonucleotide sequence functionalized to the linker molecule. The single stranded DNA oligomer is built from the 3′ end of the initiator oligonucleotide sequence. The system further includes a reading device that interprets the single stranded DNA oligomer by decoding the interpreted single stranded DNA oligomer into the set of information. The reading device comprises a molecular electronics sensor that produces distinguishable signals in a measurable electrical parameter of the molecular electronics sensor, when interpreting the single stranded DNA oligomer.

### BRIEF DESCRIPTION OF THE FIGURES

The drawings illustrate generally, by way of example, but not by way of limitation, various aspects of the present invention.

FIG. 1A is a schematic depiction of initiator particle for enzymatic DNA synthesis.

FIG. 1B is a cross sectional schematic depiction of the initiator particle for enzymatic DNA synthesis.

### DETAILED DESCRIPTION OF THE INVENTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “DNA” refers to both biological DNA molecules and synthetic versions, such as made by nucleotide phosphoramidite chemistry, ligation chemistry or other synthetic organic methodologies. DNA, as used herein, also refers to molecules comprising chemical modifications to the bases, sugar, and/or backbone, such as known to those skilled in nucleic acid biochemistry. These include, but are not limited to, methylated bases, adenylated bases, other epigenetically marked bases, and non-standard or universal bases such as inosine or 3-nitropyrrole, or other nucleotide analogues, or ribobases, or abasic sites, or damaged sites. DNA also refers expansively to DNA analogues such as peptide nucleic acids (PNA), locked nucleic acids (LNA), and the like, including the biochemically similar RNA molecule and its synthetic and modified forms. All these biochemically closely related forms are implied by the use of the term DNA, in the context of the data storage molecule used in a DNA data storage system herein. Further, the term DNA herein includes single stranded forms, double helix or double-stranded forms, hybrid duplex forms, forms containing mismatched or non-standard base pairings, non-standard helical forms such as triplex forms, and molecules that are partially double stranded, such as a single-stranded DNA bound to a oligo primer, or a molecule with a hairpin secondary structure. Generally as used herein, the term DNA refers to a molecule comprising a single-stranded component that can act as the template for a polymerase enzyme to synthesize a complementary strand therefrom.

DNA sequences as written herein, such as GATTACA, refer to DNA in the 5′ to 3′ orientation, unless specified otherwise. For example, GATTACA as written herein represents the single stranded DNA molecule 5′-G-A-T-T-A-C-A-3′. In general, the convention used herein follows the standard convention for written DNA sequences used in the field of molecular biology.

The term “polymerase” refers to an enzyme that catalyzes the formation of a nucleotide chain by incorporating DNA or DNA analogues, or RNA or RNA analogues, against a template DNA or RNA strand. The term polymerase includes, but is not limited to, wild-type and mutant forms of DNA polymerases, such as Klenow, E. Coli Pol I, Bst, Taq, Phi29, and T7, wild-type and mutant forms of RNA polymerases, such as T7 and RNA Pol I, and wild-type and mutant reverse transcriptases that operate on an RNA template to produce DNA, such as AMV and MMLV.

The term “dNTP” refers to both the standard, naturally occurring nucleoside triphosphates used in biosynthesis of DNA (i.e., dATP, dCTP, dGTP, and dTTP), and natural or synthetic analogues or modified forms of these, including those that carry base modifications, sugar modifications, or phosphate group modifications, such as an alpha-thiol modification or gamma phosphate modifications, or the tetra-, penta-, hexa- or longer phosphate chain forms, or any of the aforementioned with additional groups conjugated to any of the phosphates, such as the beta, gamma or higher order phosphates in the chain. In general, as used herein, “dNTP” refers to any nucleoside triphosphate analogue or modified form that can be incorporated by a polymerase enzyme as it extends a primer, or that would enter the active pocket of such an enzyme and engage transiently as a trial candidate for incorporation.

The terms, “binary data” or “digital data” refers to data encoded using the standard binary code, or a base 2 {0,1} alphabet, data encoded using a hexadecimal base 16 alphabet, data encoded using the base 10 {0-9} alphabet, data encoded using ASCII characters, or data encoded using any other discrete alphabet of symbols or characters in a linear encoding fashion.

The term, “digital data encoded format” refers to a series of binary digits, or other symbolic digits or characters that come from the primary translation of DNA sequence features used to encode information in DNA, or the equivalent logical string of such classified DNA features. In some aspects, information to be archived as DNA may be translated into binary, or may exist initially as binary data, and then this data may be further encoded with error correction and assembly information, into the format that is directly translated into the code provided by the distinguishable DNA sequence features. This latter association is the primary encoding format of the information. Application of the assembly and error correction procedures is a further, secondary level of decoding, back towards recovering the source information.

The term, “distinguishable DNA sequence features” means those features of a data-encoding DNA molecule that, when processed by a sensor polymerase, produce distinct signals that can be used to encode information. Such features may be, for example, different bases, different modified bases or base analogues, different sequences or sequence motifs, or combinations of such to achieve features that produce distinguishable signals when processed by a sensor polymerase.

The term, a “DNA sequence motif” refers to both a specific letter sequence or a pattern representing any member of a specific set of such letter sequences. For example, the following are sequence motifs that are specific letter sequences: GATTACA, TAC, or C. In contrast, the following are sequence motifs that are patterns: G[A/T]A is a pattern representing the explicit set of sequences {GAA, GTA}, and G[2-5] is a pattern referring to the set of sequences {GG, GGG, GGGG, GGGGG}. The explicit set of sequences in the unambiguous description of the motif, while such pattern shorthand notations as those are common compact ways of describing such sets. Motif sequences such as these may be describing native DNA bases, or may be describing modified bases, in various contexts. In various contexts, the motif sequences may be describing the sequence of a template DNA molecule, and/or may be describing the sequence on the molecule that complements the template.

The term, “sequence motifs with distinguishable signals,” in the cases of patterns, means that there is a first motif pattern representing a first set of explicit sequences, and any of said sequences produces the first signal, and there is a second motif pattern representing a second set of explicit sequences, and any of said sequences produces the second signal, and the first signal is distinguishable from the second signal. For example, if motif G[A/T]A and motif G[3-5] produce distinguishable signals, it means that any of the set {GAA, GTA} produce a first signal, and any of the set {GGG,GGGG,GGGGG} produce a second signal, distinguishable from the first.

The term, “distinguishable signals” refers to one electrical signal from a sensor being discernably different than another electrical signal from the sensor, either quantitatively (e.g., peak amplitude, signal duration, and the like) or qualitatively, (e.g., peak shape, and the like), such that the difference can be leveraged for a particular use. In a non-limiting example, two current peaks versus time from an operating molecular sensor are distinguishable if there is more than about a 1×10⁻¹⁰ Amp difference in their amplitudes. This difference is sufficient to use the two peaks as two distinct binary bit readouts, e.g., a 0 and a 1. In some instances, a first peak may have a positive amplitude, e.g., from about 1×10⁻¹⁰ Amp to about 20×10⁻¹⁰ Amp amplitude, whereas a second peak may have a negative amplitude, e.g., from about 0 Amp to about −5×10⁻¹⁰ Amp amplitude, making these peaks discernably different and usable to encode different binary bits, i.e., 0 or 1.

The term, a “data-encoding DNA molecule,” or “DNA data encoding molecule,” refers to a molecule synthesized to encode data in DNA, or copies or other DNA derived from such molecules.

As used herein, electrodes refer to nano-scale conducting metal elements, with a nanoscale-sized gap between two electrodes in an individual pair of electrodes, and, in some aspects, comprising a gate electrode capacitively coupled to the gap region, which may be a buried or “back” gate, or a side gate. The electrodes may be referred to as “source” and “drain” electrodes in some contexts, or as “positive” and “negative” electrodes, such terminologies being common in electronics. Nano-scale electrodes will have a gap width between each electrode in a pair of electrodes in the 1 nm-100 nm range, and will have other critical dimensions, such as width and height and length, also in this range. Such nano-electrodes may comprise a variety of materials that provide conductivity and mechanical stability, such as metals, or semiconductors, for example, or of a combination of such materials. Examples of metals for electrodes include titanium and chromium.

The advent of digital computing in the 20^{th} Century created the need for archival storage of large amounts of digital or binary data. Archival storage is intended to house data for long periods of time, e.g., years, decades or longer, in a way that is very low cost, and that supports the rare need to re-access the data. Although an archival storage system may feature the ability to hold unlimited amounts of data at very low cost, such as through a physical storage medium able to remain dormant for long periods of time, the data writing and recovery in such a system can be the relatively slow or otherwise costly processes. The dominant forms of archival digital data storage that have been developed to date include magnetic tape, and, more recently, compact optical disc (CD). However, as data production grows, there is a need for even higher density, lower cost, and longer lasting archival digital data storage systems.

It has been observed that in biology, the genomic DNA of living organisms functions as a form of digital information archival storage. On the timescale of the existence of a species, which may extend for thousands to millions of years, the genomic DNA in effect stores the genetic biological information that defines the species. The complex enzymatic, biochemical processes embodied in the biology, reproduction and survival of the species provide the means of writing, reading and maintaining this information archive. This observation has motivated the idea that perhaps the fundamental information storage capacity of DNA could be harnessed as the basis for high density, long duration archival storage of more general forms of digital information.

What makes DNA attractive for information storage is the extremely high information density resulting from molecular scale storage of information. In theory for example, all human-produced digital information recorded to date, estimated to be approximately 1 ZB (ZettaByte) (˜10²¹ Bytes), could be recorded in less than 10²² DNA bases, or 1/60^{th} of a mole of DNA bases, which would have a mass of just 10 grams. In addition to high data density, DNA is also a very stable molecule, which can readily last for thousands of years without substantial damage, and which could potentially last far longer, for tens of thousands of years, or even millions of years, such as observed naturally with DNA frozen in permafrost or encased in amber.

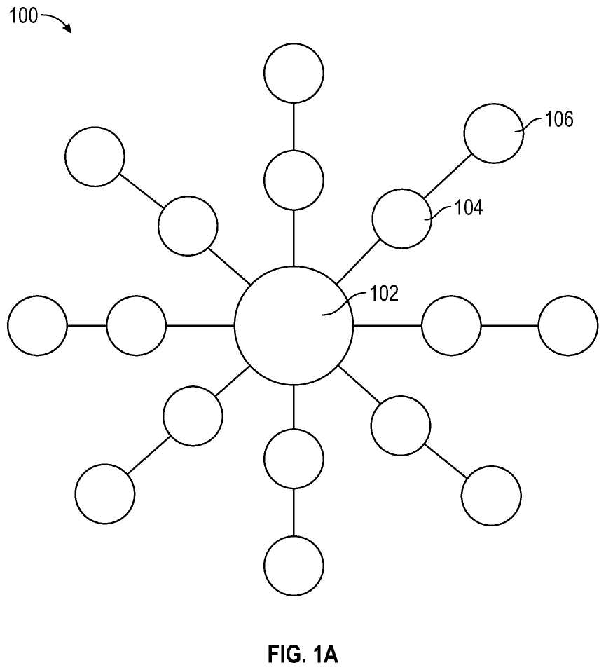

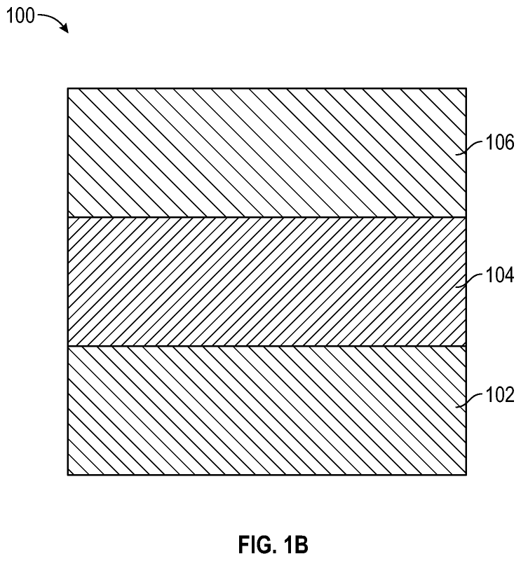

FIGS. 1A and 1B are schematic depictions of initiator particle 100. As shown, initiator particle 100 includes substrate 102, linker molecule 104, and initiator oligonucleotide 106. Substrate 102 is functionalized or bonded to linker molecule 104. Initiator oligonucleotide 106 is bonded or functionalized to linker molecule 104.

The dimensions and morphology of initiator particle 100 are largely dictated by substrate 102. Substrate 102 and thus initiator particle 100 can be a microscale particle or a nanoscale particle. For example, a largest dimension (e.g., diameter or width) of a nanoscale initiator particle 100, can be in a range of from about 0.5 nm to about 10,000 nm, about 10 nm to about 100 nm, less than, equal to, or greater than about 0.5 nm, 10, 50, 100, 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or about 10,000 nm. A largest dimension of a microscale initiator particle 100 can be in a range of from about 0.5 μm to about 10,000 μm, about 10 μm to about 100 μm, less than, equal to, or greater than about 0.5 μm, 10, 50, 100, 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or about 10,000 μm.

The morphology of initiator particle 100 can be substantially spherical, substantially cylindrical, substantially planar, conform to a nanorod structure, conform to a nanofiber structure, conform to a nanostar structure, or conform to a nanocup structure. The material of substrate 102 is silver, gold, glass, iron, or a combination thereof. The gold can be elemental gold or an alloy of gold. The silver can be elemental silver or an alloy of silver. The iron can be part of a magnetic compound. For example, the magnetic compound comprises magnetite, maghemite, or a combination thereof. The glass can include any desired glass material.

Linker molecule 104 can be functionalized to substrate 102 or to initiator oligonucleotide 106. There are various types of linker molecules 104, that can be used. Examples of suitable linker molecules 104 include streptavidin, a biotin, a thiol, or a combination thereof. The streptavidin can be monovalent, divalent, or trivalent. Biotin is classified as a heterocyclic compound, with a sulfur-containing ring fused ureido and tetrahydrothiophene group. A C5-carboxylic acid side chain is appended to one of the rings. The ureido ring, containing the —N—CO—N— group, serves as the carbon dioxide carrier in carboxylation reactions.

Biotin is a coenzyme for five carboxylase enzymes, which are involved in the catabolism of amino acids and fatty acids, synthesis of fatty acids, and gluconeogenesis. Biotinylation of histone proteins in nuclear chromatin plays a role in chromatin stability and gene expression.

A thiol or thiol derivative, is any organosulfur compound of the form R—SH, where R represents an alkyl or other organic substituent. The —SH functional group itself is referred to as either a thiol group or a sulfhydryl group, or a sulfanyl group. Thiols are the sulfur analogue of alcohols (that is, sulfur takes the place of oxygen in the hydroxyl (—OH) group of an alcohol), and the word is a blend of “thio-” with “alcohol”.

Additional linkers can include an amine. For instance, the amine can be a primary or secondary amine present on a molecule.

In some examples where substrate 102 includes gold or silver, linker molecule 104 can be a thiol. Similarly, in some examples, where substrate 102 includes glass linker molecule 104 can be a thiol. In some further examples where substrate 102 includes iron, linker molecule 104 can include streptavidin, biotin, or a mixture thereof. In some further examples, substrate 102 may not be modified with linker molecule 104, but rather initiator oligonucleotide sequence 106 can be modified with linker molecule 104. In that case, initiator oligonucleotide sequence 106 is modified with a thiol group functionalized to the 5′ end of the oligonucleotide sequence. The specific choice of linker molecule 104 will depend on which substrate 102 is used.

Initiator oligonucleotide 106 is ultimately functionalized to substrate 102 by linker molecule 104. As shown in FIG. 1, there are a plurality of initiator oligonucleotides 106 functionalized to substrate 102. In various examples, about 30% to about 100% total surface area of substrate 102 is functionalized with the plurality of initiator oligonucleotides, about 70% to about 95% total surface area, less than, equal to, or greater than about 30% total surface area, 35, 40, 45, 50, 55, 60, 65, 70, 75, 80, 85, 90, 95, or about 100% total surface area.

The plurality of initiator oligonucleotides 106 can have at least 95% sequence identity with respect to each other or at least 99% sequence identity with respect to each other. Alternatively, at least two initiator oligonucleotides 106 can be very different from each other (e.g., share less than 90% sequence identity). Such a distribution of initiator oligonucleotides 106 can be helpful if different coded oligonucleotides are desired to be synthesized using initiator particle 100.

A coded oligomer sequence can be built on initiator particle 100. Synthesis of the coded oligomer sequence is carried out on initiator oligomer 106. The coded oligomer sequence can be a single stranded DNA oligomer. The coded oligomer sequence can include any suitable number of nucleotides needed to encode the desired information. For example, the coded oligomer sequence can include 10 to 90 bases, 20 to 80 bases, less than, equal to, or greater than 10 bases, 15, 20, 25, 30, 35, 40, 45, 50, 55, 60, 65, 70, 75, 80, 85, or 90 bases.

Initiator particle 100 can be used in conjunction with many different DNA synthesis devices. Examples of DNA synthesis devices include, without limitation, a microarray. DNA synthesis devices can be automated devices.

Depending on the type of DNA synthesis device, initiator particle 100 can be disposed in various types of reaction chambers. A reaction chamber can be a channel, a well (e.g., if the device is a microarray), a cartridge, a pore, or reaction site. DNA synthesis devices can include as few as one reaction chamber that includes initiator particle 100. In other examples, a DNA synthesis device can include any plural number of reaction chambers at least some of which or all can include initiator particle 100. For example, a DNA synthesis device can include 1 to 500 reaction chambers 108, 5 to 250 reaction chambers, 40 to 100 reaction chambers, less than, equal to, or greater than 2 reaction chambers, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500. Where multiple reaction chambers are present, each reaction chamber can have substantially the same dimensions. Alternatively, at least two reaction chambers may differ in their dimensions. For example, one reaction chamber may be shaped as a well, whereas another reaction chamber may be formed as a channel.

Each reaction chamber can include the same initiator particle 100 or a distribution of different initiator particles. Initiator particles 100 can be “different” by way of the material of substrate 102, type of linker molecule 104, initiator oligomer 106, the coded oligomer, or a mixture thereof. The coded oligomer represents a desired sequence. For each desired sequence, multiple DNA molecules representing that sequence are produced on initiator particle 100. The multiplicity of molecules produced can be in the ranges of 10's, 100's, 1000's, millions or even billions of copies of DNA molecules for each desired sequence. All of these copies representing all the desired sequences may be pooled into one master pool of molecules. It is typical of such DNA writing systems that the writing is not perfect, and if N molecules are synthesized to represent a given input sequence, not all of these will actually realize the desired sequence. For example, they may contain erroneous deletions, insertions, or incorrect or physically damaged bases.

The sequence of nucleotide oligomer 106 can be determined by an encoder/decoder, which includes an algorithm with two functions: the encoder portion translates given digital data encoded format (e.g., digital/binary information) into a specific set of DNA sequences that are inputs to the DNA writer. The decoder portion translates a given set of DNA sequences of the type provided by the DNA reader, back into digital information.

The nucleotide oligomer can bases chosen from adenine, cytosine, guanine, and thymine. When in their reactant form (e.g., before polymerizing to form the oligomer), individual nucleotides are dNTPs. A polymerase used for synthesis of the nucleotide oligomer 106 can be a native or mutant form of Klenow, Taq, Bst, Phi29 or T7, or may be a reverse transcriptase. In various aspects, the mutated polymerase forms will enable site specific conjugation of the polymerase to the bridge molecule, arm molecule or electrodes, through introduction of specific conjugation sites in the polymerase. Such conjugation sites engineered into the protein by recombinant methods or methods of synthetic biology may, in various aspects, comprise a cysteine, an aldehyde tag site (e.g. the peptide motif C×P×R), a tetracysteine motif (e.g., the peptide motif CCPGCC) (SEQ ID NO: 1), or an unnatural or non-standard amino acid (NSAA) site, such as through the use of an expanded genetic code to introduce a p-acetylphenylalanine, or an unnatural cross-linkable amino acid, such as through use of RNA- or DNA-protein cross-link using 5-bromouridine, (see, e.g., Gott, J. M., et al., Biochemistry, 30 (25), pp 6290-6295 (1991)).

The information encoder/decoder used to select the nucleotides to correspond to the data to be encoded can be selected based on the properties of the DNA writer and DNA reader devices, so as to minimize or reduce some overall measure of the cost of the information storage/retrieval process. One key component of system cost is the overall error rate in retrieved information.

In general, a DNA writer device can introduce writing errors, and a DNA reading device can produce reading errors, and so the processes of storing information in the system and then later retrieving it potentially results in an error rate seen in the retrieved information. The encoder/decoder algorithm can be chosen to minimize or reduce this error rate, based on the error properties and propensities of the DNA reader and DNA writer.

In various aspects, nucleotides can be preferentially selected for incorporation in nucleotide sequences based on their ease of synthesis in the writing process that forms molecules, reduced propensity to form secondary structure in the synthesized molecules, and/or ease in reading during the data decoding process. In various aspects, bad writing motifs and bad reading motifs are avoided in the selection of nucleotides for incorporation into nucleotide sequences, with a focus on incorporating segments in the nucleotide sequence that will produce mutually distinguishable signals when that nucleotide sequence is read to decode the encoded information. For example, in reading a nucleotide sequence, A and T are mutually distinguishable, C and G are mutually distinguishable, A, C and G are mutually distinguishable, AAA and TT are mutually distinguishable, A, GG and ATA are mutually distinguishable, and C, G, AAA, TTTT, GTGTG are mutually distinguishable. These, and many other sets of nucleotide and nucleotide segments provide mutually distinguishable signals in a reader, and thus can be considered for incorporation in a nucleotide sequence when encoding a set of information into a nucleotide sequence.

Additionally, there are nucleotide segments that are difficult to write, and thus should be avoided when encoding a set of information into a nucleotide sequence. In various aspects, encoding of a set of information into a nucleotide sequence comprises the use of one of the remaining distinguishable feature sets as the encoding symbols, such as may correspond to binary 0/1, trinary 0/1/2 or quad 0/1/2/3 code, etc., along with an error correcting encoding to define the set of information in a way that avoids the hard to read and hard to write features. In this way, overall performance of an information storage system is improved.

In general, in order to reduce errors, the digital data encoding/decoding algorithm can comprise error detecting and error correcting codes selected to minimize error production, given the actual error modes of the DNA writer and DNA reader. These codes can be devised with the benefit of prior knowledge of the error modes, i.e., the propensity for particular errors of the writer and reader.

In various aspects, the error correcting codes reside within a single nucleotide sequence. For example, one segment of binary data is encoded in one DNA sequence, with the use of error correction and/or detection schemes on the DNA side. Such schemes may also involve encoding one segment of binary data into multiple DNA sequences, to provide another level of redundant encoding of information, which is analogous to error correction through redundant storage. Error detection schemes include, but are not limited to, repetition code, parity bits, checksums, cyclic redundancy checks, cryptographic hash functions, and error correcting codes such as hamming codes. Error correction schemes include, but are not limited to, automatic repeat request, error correcting code such as convolutional codes and block codes, hybrid automatic repeat request, and Reed-Solomon codes.

In various aspects, a method of devising an optimal or highly efficient error correcting encoding, wherein the incoming digital data is considered as binary words of length N, comprises the steps of: providing a space of all DNA words of length M, such that there are many more possible DNA words than binary words (i.e., 4^{M}>>2^{N}); and selecting a subset of 2^{N} of the DNA words to use as code words for encoding the 2^{N} binary information words, such that when each of these DNA code words is expanded into the set of probable DNA writing errors for the given word, and then that set further expanded by the set of probable reading errors words, these resulting 2^{N} sets of DNA words remain disjoint with high probability. In such a case, any word read by the reader can be properly associated back to the ideal encoded DNA word with very high probability. This method constitutes a combination of error correcting and error avoiding encoding of information. In addition, the decoding algorithm would also naturally make use of confidence or odds information supplied by the reader, to select the maximum likelihood/highest confidence decoding relative the encoding scheme.

Following synthesis of the coded oligomer, the coded oligomer can be cleaved from initiator particle 100. This can be accomplished by cleaving the bond between initiator particle 100 and the coded oligomer. Following cleaving, initiator particle 100 can be recovered to be used in a subsequent DNA synthesis procedure. However, in some examples, the coded oligomer may not be cleaved immediately. This can be the case where the coded oligomer is “stored” on initiator particle 100. In some examples, if a mixture of different coded oligomers are stored together, the properties of substrate 102 may help to access a desired coded oligomer. For example, if a first coded oligomer is stored on a substrate 102 having a first mass and a second coded substrate is stored on a second substrate 102, having a second mass that is different than the first mass, the respective initiator particles 100 can be separated using centrifugation. As another example some substrates 102 can include iron so that various initiator particles 100 can be separated using magnetics. Different substrates 102 can also have different densities, which can be used to separate initiator particles 100.

The coded oligomer can be read using a DNA reader device. In various aspects, the reading device for use herein is based on a CMOS chip sensor array device in order to increase the speed and scalability and decrease the capital costs. An aspect of such a device comprises a CMOS sensor array device, wherein each sensor pixel contains a molecular electronic sensor capable of reading a single molecule of DNA without any molecular amplification or copying, such as PCR, required. In various aspects, the CMOS chip comprises a scalable pixel array, with each pixel containing a molecular electronic sensor, and such a sensor comprising a bridge molecule and polymerase enzyme, configured so as to produce sequence-related modulations of the electrical current (or related electrical parameters such as voltage, conductance, etc.) as the enzyme processes the DNA template molecule.

As an example, the coded oligomer can be read with a DNA reading device, which is a device that takes a pool of DNA molecules and produces a set of measured DNA sequences for molecules sampled or selected from this pool. Such readers actually survey only a small portion of the DNA molecules introduced into the system, so that only a small fraction will undergo an actual read attempt. It is further typical of such DNA reading devices that a given DNA molecule that is processed may not be read with entire accuracy, and thus there may be errors present in the read. As a result, it is also typical that the measured sequence outputs include various forms of confidence estimates and missing data indicators. For example, for each letter in a measure sequence, there may be a confidence probability or odds that it is correct, versus the other three DNA letter options, and there may be missing data indicators that indicate the identity of a letter is unknown, or there may be a set of optional sequence candidates with different probabilities representing a portion of a read.

Another key aspect of optimizing the overall DNA data storage system costs is the time required to write data. For example, the critical time cost in many aspects may be the time cost of writing the data. In various aspects, the writing of certain slow-to-synthesize bases and sequence motifs are avoided in order to shorten the overall writing time. In other aspects, the writing is faster, such as by reducing the time spent on each chemistry cycle of some cyclical process that writes one base in many parallel synthesis reactions, with acceptance of a higher overall writing error rate.

Similarly, for reading, a faster reading process may be employed, with the trade-off being a higher rate of reading errors. In various examples, a faster reading process is employed without an increase in error by avoiding the introduction of certain types of sequences in the encoding that are difficult to read at a rapid rate, such as homopolymer runs. In either case, the information encoding/decoding algorithm can be co-optimized with these choices that allow for faster reading/writing but with extra error modes to be avoided, or avoiding slow-to-read/write sequence motifs, handled within the encoding/decoding.

In various aspects of the DNA information storage system herein, the DNA reading device comprises a massively parallel DNA sequencing device, which is capable of a high speed of reading bases from each specific DNA molecule such that the overall rate of reading stored DNA information can be fast enough, and at high enough volume, for practical use in large scale archival information retrieval. The rate of reading bases sets a minimum time on data retrieval, related to the length of stored DNA molecules.

In various aspects, a molecular electronics sensor extracts information from single DNA molecules, in a way that provides a reader for digital data stored as DNA. A molecular electronics sensor comprises a circuit in which a single molecule, or a complex of a small number of molecules, forms a completed electrical circuit spanning the gap between a pair of nano-scale electrodes, and an electronic parameter is modulated by this single molecule or complex, and in which this parameter is measured as a signal to indicate (“sense”) the single molecule or complex interacting with target molecules in the environment. In various aspects, the measured parameter is current passing through the electrodes, versus time, and the molecular complex is conjugated in place with specific attachment points to the electrodes.

A cloud based DNA data archival storage system can be used in conjunction with microscale or nanoscale system 100, in which the complete reader system in certain aspects, deployed in aggregate format to provide the cloud DNA reader server of the overall archival storage and retrieval system. A cloud computer system can include a standard storage format. Such as standard cloud computer system comprises a DNA archival data storage capability as indicated. In various aspects, a cloud-based DNA synthesis system can accept binary data from the cloud computer and produce the physical data encoding DNA molecules. This server stores the output molecules in a DNA data storage archive wherein the physical DNA molecules that encode data are stored in a dried or lyophilized form, or in solution, at ambient temperature, cooled temperature, or frozen. When data is to be retrieved, a sample of the DNA from the archive is provided to the DNA data reader server, which outputs decoded binary data back to the primary cloud computer system. This DNA data reader server is, in certain aspects, powered by a multiplicity of DNA reader chip-based systems in combination with additional computers that perform the final decoding of the DNA derived data back to the original data format of the primary cloud storage system.

Search of an archive for a literal input string can be achieved by encoding the search string or strings of interest into DNA form, synthesizing a complementary form or related primers for the desired DNA sequences, and using hybridization or PCR amplification to assay the archive for the presence of these desired sequence fragments, according to such standard assays are used by those skilled in the art of molecular biology to ascertain the presence of a sequence segment in a complex pool of DNA fragments. The search could report either presence or absence, or could recover the associated fragments containing the search string for complete reading.

### EXAMPLES

Various aspects of the present invention can be better understood by reference to the following Examples which are offered by way of illustration. The present invention is not limited to the Examples given herein.

### Example 1: Definition of Initiator Oligonucleotide Sequence for Surface Modification and Conjugation with Gold Nanoparticles

The strategy used in the definition of the oligonucleotide sequences was the application of standardized commercial sequences of the M13 type, of 18 bases, added with 10 bases A at 5, which do not contain biological significance, commonly used in the amplification of plasmid regions by PCR, extensively applied in the routine of molecular biology laboratories. The sequence is described below:

Initiator M13 (SEQ ID NO: 1);
Artificial nucleotide sequence initiator:
HS-AAA AAA AAA ATG TAA AAC GAC GGC CAG T

The sequences were synthesized at the 50 nmol scale, containing the modification Thiol C6 at the 5′ end. In FIG. 1 the circle illustrates the gold nanoparticles and the lines on the surface represent the initiators attached to the surface of the nanoparticle, by a sulfur bound.

### Example 2: Gold Nanoparticle Synthesis (AuNP)

200 mL of suspension of gold nanoparticles (AuNP) with an average size of 26 nm were synthesized in a 500 mL glass reactor with a Teflon impeller. It was mixed 20 mL of auric chloride solution (10 mM) with 160 ml of nuclease-free water. The mixture was heated to 95° ° C. under controlled stirring at 950 rpm. After reaching the maximum temperature, 20 mL of sodium citrate solution (25 mM) was quickly added, keeping the agitation at 950 rpm for 25 minutes; then the reactor temperature was set to 25° C. and kept under constant stirring at 950 rpm for another 15 minutes. At the end, the AuNP suspension was stored in a refrigerator at an average temperature of 8° C.

The synthesis was characterized by UV spectrophotometry, scanning from 200 to 850 nm to verify the highest absorbance peak at 526 nm and estimate the average size in nanometers.

### Example 3: Conjugation of Oligonucleotides with Gold Nanoparticles

The conjugation step consisted of linking the initiator with the gold nanoparticles. Each DNA molecule can covalently bind, via the thiol moiety, to only a single gold particle. However, each gold particle can accommodate many DNA molecules. Given this, a protocol was tested with several ratios of DNA molecules for each gold particle. The different proportions tested were as follows: 0, 50, 80, 150, 300, 500, 750, 1000 and 1500 times more DNA units than gold particles.

The conjugation process was carried out using 2 mL of suspension of AuNP with a concentration of 1 mM of Au atoms and an average size of 20 nm, to which 36.5 μL of initiator M13 (SEQ ID NO: 1) at a concentration of 50 micromolar to obtain a ratio of 1 particle to 900 strands of DNA. After 10 minutes of stirring at 350 rpm at room temperature, 10 μL of sodium citrate solution (500 mM, pH 3.0) and 5 μL of hydrochloric acid solution (1 M) were added to adjust the pH to 3. After this step, the solution was stirred at 350 rpm for 20 minutes and then centrifuged at 14,000 rpm for 15 minutes. The supernatant was then discarded and the pellet was resuspended with 2 mL of HEPES buffer (10 mM).

The suspension obtained is characterized as a composite solution, ready to be used as the solid material to the DNA synthesis. It has the visual appearance of a liquid, clear, pink to wine color and maximum absorption peak at UV-Vis of 530 nm.

### Example 4: Synthesis and Elongation of DNA Molecule on the Surface of Nanoparticles with an Attached Pre-Built Block

The samples were prepared in a 200 μL microtube with a volume of 15.5 μL of the composite suspension obtained on example 3, 2 μL ZnCl₂, 2 μL dNTP 100 mM (5000:1 ratio between DNA and nucleotides), 0.5 L terminal deoxynucleotidyl transferase (TdT) and left for 20 minutes or 1 hour at 37° C. To stop the reaction, the solution was immediately mixed with a loading dye and put into the wells of an agarose gel. An 80V current ran through the box for 45 minutes. The gel was transferred to a plate and photo documented revealing that with each extension reaction.

| Sample | Reaction time (min) |
|---|---|
| Control | AuNP + DNA not elongated |
| 1 | 20 min |
| 2 | 20 min |
| 3 | 20 min |
| 4 | 60 min |
| 5 | 60 min |
| 6 | 60 min |

### Example 5: Synthesis and Elongation of a Determined DNA Molecule on the Surface of Nanoparticles with an Attached Pre-Built Block

The samples were prepared in a 200 μL microtube with a volume of 13.8 μL of the composite suspension obtained on example 3, 2 μL CoCl₂, 2 μL dATP (5000:1 ratio between DNA and nucleotides), 0.2 L terminal deoxynucleotidyl transferase (TdT) and left for 30 minutes at 37° C. To stop the reaction, the solution was incubated at 70° C. for 10 minutes. The microtube was centrifuged at 14,000 rotations per minute (rpm) for 15 minutes. The supernatant was removed and the gold particles were resuspended in 13.8 μL water. By the end of the reaction there were M13 DNA molecules, bonded on the AuNP surface, with a polyA added in the sequence, M13+A₍ₙ₎.

In the same tube which the gold nanoparticles was resuspended, it were prepared a new synthesis reaction, composed of 2 μL CoCl₂, 2 μL dCTP (5000:1 ratio between DNA and nucleotides), 0.2 μL TdT enzyme, and the reaction was incubated for 30 minutes at 37° C. To stop the reaction, it was incubated at 70° C. for 10 minutes. The microtube was centrifuged at 14,000 rpm for 15 minutes. The supernatant was removed and the gold particles were resuspended in 13.8 μL water. By the end of the reaction there were DNA molecules, bonded to the particles, with the sequence M13+A₍ₙ₎+C₍ₙ₎.

The exact same procedure was repeated as above, but using the T, and G nucleotides. By changing the nucleotide base in each reaction, it was possible to control the sequence being synthetized. By the end of the four reactions there was DNA molecules, bind to the bead, with the sequence M13+A₍ₙ₎+C₍ₙ₎+T₍ₙ₎+G₍ₙ₎, in that order.

An agarose gel was prepared by diluting 1 g of agarose into 100 ml of Tris-Acetate-EDTA (TAE). This solution was heated until melting and dissolving the whole agarose. Adds 10 μL of SYBR safe and pour the liquid into a mold, with a comb on top. The gel polymerases in the shape of the mold, that solidified, was placed into the electrophoresis unit or box, which was filled with TAE. The samples were mixed with a loading dye and put into the wells of the gel using a pipette. An 80V current ran through the box for 45 minutes. The gel was transferred to a plate and photo documented revealing that with each extension reaction, longer DNA strands were seen in the sample.

| Sample | Feature |
|---|---|
| 1 | 20 min |
| 2 | 20 min |
| 3 | M13 + A₍ₙ₎ |
| 4 | M13 + A₍ₙ₎ + C₍ₙ₎ |
| 5 | M13 + A₍ₙ₎ + C₍ₙ₎ + T₍ₙ₎ |
| 6 | M13 + A₍ₙ₎ + T₍ₙ₎ + C₍ₙ₎ + G₍ₙ₎ |

### Example 5: Iron Oxide Nanoparticle Synthesis

First, the synthesis of iron oxide was carried out by coprecipitation. In a three-necked round-bottom flask, 1.99 g of FeCl2·4H2O and 4.05 g of FeCl₃·6H₂O in 150 mL of distilled water, subjected to ultrasound application, mechanical agitation at 400 rpm and nitrogen bubbling. After 5 minutes, the dripping of 50 mL of NH₃ solution (25%) was started, which lasted 10 min. After another 40 min under the same conditions, N₂, ultrasound and mechanical agitation were stopped. The precipitate was separated from the medium by a magnet and the supernatant was discarded. Centrifugation at 8000 rpm for 10 min was performed and the supernatant was discarded once more. The precipitate was resuspended in 500 ml of water.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Aspects

The following exemplary aspects are provided, the numbering of which is not to be construed as designating levels of importance:

Aspect 1 provides an initiator particle for enzymatic DNA synthesis, the initiator particle comprising:
a substrate comprising:
   silver;
   gold;
   glass;
   iron; or
   a combination thereof;
a linker molecule functionalized to the substrate; and
an initiator oligonucleotide sequence functionalized to the linker molecule.

Aspect 2 provides the initiator particle of Aspect 1, wherein the initiator particle is a microscale particle or a nanoscale particle.

Aspect 3 provides the initiator particle of any of Aspects 1 or 2, wherein the initiator particle size is in a range of from about 0.5 nm to about 10,000 nm.

Aspect 4 provides the initiator particle of Aspect 3, wherein the initiator particle size is in a range of from about 10 nm to about 100 nm.

Aspect 5 provides the initiator particle of any of Aspects 1 or 2, wherein the initiator particle size is in a range of from about 0.5 μm to about 10,000 μm.

Aspect 6 provides the initiator particle of Aspect 5, wherein the initiator particle size is in a range of from about 10 μm to about 100 μm.

Aspect 7 provides the initiator particle of any of Aspects 1-6, wherein the initiator particle is substantially spherical, substantially cylindrical, substantially planar, conform to a nanorod structure, conform to a nanofiber structure, conform to a nanostar structure, or conform to a nanocup structure.

Aspect 8 provides the initiator particle of any of Aspects 1-7, wherein the gold is elemental gold or an alloy of gold.

Aspect 9 provides the initiator particle of any of Aspects 1-8, wherein the iron is part of a magnetic compound.

Aspect 10 provides the initiator particle of Aspect 9, wherein the magnetic compound comprises magnetite, maghemite, or a combination thereof.

Aspect 11 provides the initiator particle of any of Aspects 1-10, wherein the silver is elemental silver or an alloy of silver.

Aspect 12 provides the initiator particle of any of Aspects 1-11, wherein the linker molecule comprises streptavidin, a biotin, a thiol, an amine, or a combination thereof.

Aspect 13 provides the initiator particle of any of Aspects 1-12, wherein the substrate is gold and the linker molecule is a thiol.

Aspect 14 provides the initiator particle of any of Aspects 1-13, wherein the substrate is glass and the linker molecule is a thiol.

Aspect 15 provides the initiator particle of any of Aspects 1-14, wherein the substrate comprises iron and the linker molecule comprises streptavidin, biotin, or a mixture thereof.

Aspect 16 provides the initiator particle of any of Aspects 1-15, wherein the initiator oligonucleotide sequence is modified with a thiol group functionalized to the 5′ end of the oligonucleotide sequence.

Aspect 17 provides the initiator particle of any of Aspects 1-16, wherein the substrate comprises a plurality of initiator oligonucleotides functionalized thereto.

Aspect 18 provides the initiator particle of Aspect 17, wherein each of the plurality of the initiator oligonucleotides comprise at least 95% sequence identity with respect to each other.

Aspect 19 provides the initiator particle of any of Aspects 17 or 18, wherein each of the plurality of the initiator oligonucleotides comprise about 99% sequence identity with respect to each other.

Aspect 20 provides the initiator particle of any of Aspects 17 or 19, wherein about 30% to about 100% total surface area of the substrate is functionalized with the plurality of initiator oligonucleotides.

Aspect 21 provides the initiator particle of any of Aspects 17-20, wherein about 70% to about 95% total surface area of the substrate is functionalized with the plurality of initiator oligonucleotides.

Aspect 22 provides the initiator particle of any of Aspects 1-21, further comprising a coded oligomer sequence bonded to the initiator oligonucleotide.

Aspect 23 provides the initiator particle of Aspect 22, wherein the coded oligomer sequence comprises 10 to 90 bases.

Aspect 24 provides the initiator particle of any of Aspects 22 or 23, wherein the coded oligomer sequence comprises 20 to 80 bases.

Aspect 25 provides the initiator particle of any of Aspects 22-24, wherein the coded oligomer sequence is a single stranded DNA oligomer.

Aspect 26 provides an initiator particle for enzymatic DNA synthesis, the initiator particle comprising:
a substrate comprising:
   silver;
   gold;
   glass;
   iron; or
   a combination thereof;
a linker molecule functionalized to the substrate;
an initiator oligonucleotide sequence functionalized to the linker molecule; and
   a coded oligomer sequence bonded to the initiator oligonucleotide.

Aspect 27 provides a method of making the initiator particle of any of Aspects 1-26, the method comprising:
forming a reaction mixture including the substrate with the initiator oligonucleotide, wherein the substrate or the oligonucleotide have the linker molecule functionalized thereto; and
conjugating the substrate and the initiator oligonucleotide.

Aspect 28 provides the method of Aspect 27, wherein about 10 to about 1500 times more initiator oligonucleotides are added for every substrate in the reaction mixture.

Aspect 29 provides the method of any of Aspects 27 or 28, wherein about 80 to about 1000 times more initiator oligonucleotides are added for every substrate in the reaction mixture.

Aspect 30 provides a method of synthesizing a single stranded DNA oligomer, the method comprising:
contacting the initiator of any one of Aspects 1-29, with a nucleotide and an enzyme; and
building the single stranded DNA oligomer from the 3′ end of the initiator oligonucleotide sequence.

Aspect 31 provides the method of Aspect 30, wherein the enzyme comprises a polymerization enzyme.

Aspect 32 provides the method of Aspect 31, wherein the polymerization enzyme comprises a DNA polymerase.

Aspect 33 provides the method of Aspect 32, wherein the DNA polymerase is terminal deoxynucleotidyl transferase.

Aspect 34 provides the method of any of Aspects 31-33, wherein the nucleotide comprises a deoxynucleoside triphosphate.

Aspect 35 provides the method of any of Aspects 31-34, wherein the single stranded DNA oligomer comprises a set of information.

Aspect 36 provides the method of Aspect 35, wherein the set of information is binary.

Aspect 37 provides the method of any of Aspects 30-36, further comprising cleaving a bond between the initiator oligomer nucleotide and the single stranded DNA oligomer; initiator oligomer nucleotide and the imitator particle; or a combination thereof.

Aspect 38 provides a DNA synthesis device comprising:
a reaction chamber; and
the initiator particle of any of Aspects 1-37, located at least partially within the reaction chamber.

Aspect 39 provides the device of Aspect 38, further comprising:
a second reaction chamber; and
a second substrate of any of Aspects 1-38, located at least partially within a second reaction chamber.

40 provides the device of any of Aspects 38 or 39, wherein the reaction chamber and the second reaction chamber are independently a well, a channel, a cartridge, a pore, or reaction site.

Aspect 41 provides the device of any of Aspects 38-40, wherein the device is a microdevice or a nanodevice.

Aspect 42 provides the device of any of Aspects 38-41, wherein the device is a microarray.

Aspect 43 provides the device of any of Aspects 38-42, wherein the device is an automated device.

Aspect 44 provides an information storage system, comprising:
a device that synthesizes a single stranded DNA oligomer that encodes a set of information, the device comprising the initiator particle of any of Aspects 1-43; and
a reading device that interprets the single stranded DNA oligomer by decoding the interpreted single stranded DNA oligomer into the set of information,
wherein the reading device comprises a molecular electronics sensor that produces distinguishable signals in a measurable electrical parameter of the molecular electronics sensor, when interpreting the single stranded DNA oligomer.

Aspect 45 provides the system of Aspect 44, wherein the set of information is binary.

Aspect 46 provides the system of Aspect 44 or 45, further comprising at least one of error detecting schemes or error correction schemes for minimizing errors within the single stranded DNA oligomer.

Aspect 47 provides the system of Aspect 46, wherein the error detecting schemes are selected from repetition code, parity bits, checksums, cyclic redundancy checks, cryptographic hash functions and hamming codes, and the error correction schemes are selected from automatic repeat request, convolutional codes, block codes, hybrid automatic repeat request and Reed-Solomon codes.

Aspect 48 provides the system of any of Aspects 44-47, wherein the device comprises a CMOS chip based array of actuator pixels for DNA synthesis, the actuator pixels directing voltage/current or light-mediated deprotection within an enzymatic DNA synthesis reaction comprising phosphoramidite chemistries.

What is claimed is:

1. An initiator particle for enzymatic DNA synthesis, the initiator particle comprising:
a substrate comprising:
silver;
gold;
glass;
iron; or
a combination thereof;
a linker molecule functionalized to the substrate; and
an initiator oligonucleotide sequence functionalized to the linker molecule.

2. The initiator particle of claim 1, wherein the initiator particle is a microscale particle or a nanoscale particle.

3. The initiator particle of any of claim 1, wherein the initiator particle size is in a range of from about 0.5 nm to about 10,000 nm.

4. The initiator particle of claim 3, wherein the initiator particle size is in a range of from about 10 nm to about 100 nm.

5. The initiator particle of any of claim 1, wherein the initiator particle size is in a range of from about 0.5 μm to about 10,000 μm.

6. The initiator particle of claim 5, wherein the initiator particle size is in a range of from about 10 μm to about 100 μm.

7. The initiator particle of any of claim 1, wherein the initiator particle is substantially spherical, substantially cylindrical, substantially planar, conform to a nanorod structure, conform to a nanofiber structure, conform to a nanostar structure, or conform to a nanocup structure.

8. The initiator particle of claim 1, wherein the gold is elemental gold or an alloy of gold.

9. The initiator particle of claim 1, wherein the iron is part of a magnetic compound.

10. The initiator particle of claim 9, wherein the magnetic compound comprises magnetite, maghemite, or a combination thereof.

11. The initiator particle of claim 1, wherein the silver is elemental silver or an alloy of silver.

12. The initiator particle of claim 1, wherein the linker molecule comprises streptavidin, a biotin, a thiol, an amine, or a combination thereof.

13. The initiator particle of claim 1, wherein the substrate is gold and the linker molecule is a thiol.

14. The initiator particle of claim 1, wherein the substrate is glass and the linker molecule is a thiol.

15. The initiator particle of claim 1, wherein the substrate comprises iron and the linker molecule comprises streptavidin, biotin, or a mixture thereof.

16. The initiator particle of claim 1, wherein the initiator oligonucleotide sequence is modified with a thiol group functionalized to the 5′ end of the oligonucleotide sequence.

17. The initiator particle of claim 1, wherein the substrate comprises a plurality of initiator oligonucleotides functionalized thereto.

18. The initiator particle of claim 17, wherein each of the plurality of the initiator oligonucleotides comprise at least 95% sequence identity with respect to each other.

19. The initiator particle of claim 17, wherein each of the plurality of the initiator oligonucleotides comprise about 99% sequence identity with respect to each other.

20. The initiator particle of claim 17, wherein about 30% to about 100% total surface area of the substrate is functionalized with the plurality of initiator oligonucleotides.

21. The initiator particle of claim 17, wherein about 70% to about 95% total surface area of the substrate is functionalized with the plurality of initiator oligonucleotides.

22. The initiator particle of claim 1, further comprising a coded oligomer sequence bonded to the initiator oligonucleotide.

23. The initiator particle of claim 22, wherein the coded oligomer sequence comprises 10 to 90 bases.

24. The initiator particle of claim 22, wherein the coded oligomer sequence comprises 20 to 80 bases.

25. The initiator particle of claim 22, wherein the coded oligomer sequence is a single stranded DNA oligomer.

26. An initiator particle for enzymatic DNA synthesis, the initiator particle comprising:
a substrate comprising:
silver;
gold;
glass;
iron; or
a combination thereof;
a linker molecule functionalized to the substrate;
an initiator oligonucleotide sequence functionalized to the linker molecule; and
a coded oligomer sequence bonded to the initiator oligonucleotide.

27. A method of making the initiator particle of claim 1, the method comprising:
forming a reaction mixture including the substrate with the initiator oligonucleotide, wherein the substrate or the oligonucleotide have the linker molecule functionalized thereto; and
conjugating the substrate and the initiator oligonucleotide.

28. The method of claim 27, wherein about 10 to about 1500 times more initiator oligonucleotides are added for every substrate in the reaction mixture.

29. The method of claim 27, wherein about 80 to about 1000 times more initiator oligonucleotides are added for every substrate in the reaction mixture.

30. A method of synthesizing a single stranded DNA oligomer, the method comprising:
contacting the initiator of claim 1, with a nucleotide and an enzyme; and
building the single stranded DNA oligomer from the 3′ end of the initiator oligonucleotide sequence.

31. The method of claim 30, wherein the enzyme comprises a polymerization enzyme.

32. The method of claim 31, wherein the polymerization enzyme comprises a DNA polymerase.

33. The method of claim 32, wherein the DNA polymerase is terminal deoxynucleotidyl transferase.

34. The method of claim 31, wherein the nucleotide comprises a deoxynucleoside triphosphate.

35. The method of claim 31, wherein the single stranded DNA oligomer comprises a set of information.

36. The method of claim 35, wherein the set of information is binary.

37. The method of claim 30, further comprising cleaving a bond between the initiator oligomer nucleotide and the single stranded DNA oligomer; initiator oligomer nucleotide and the imitator particle; or a combination thereof.

38. A DNA synthesis device comprising:
a reaction chamber; and
the initiator particle of claim 1, located at least partially within the reaction chamber.

39. The device of claim 38, further comprising:
a second reaction chamber; and
a second substrate of any of claims 1-38, located at least partially within a second reaction chamber.

40. The device of claim 38, wherein the reaction chamber and the second reaction chamber are independently a well, a channel, a cartridge, a pore, or reaction site.

41. The device of claim 38, wherein the device is a microdevice or a nanodevice.

42. The device of claim 38, wherein the device is a microarray.

43. The device of claim 38, wherein the device is an automated device.

44. An information storage system, comprising:
a device that synthesizes a single stranded DNA oligomer that encodes a set of information, the device comprising the initiator particle of any of claims 1-43; and
a reading device that interprets the single stranded DNA oligomer by decoding the interpreted single stranded DNA oligomer into the set of information,
wherein the reading device comprises a molecular electronics sensor that produces distinguishable signals in a measurable electrical parameter of the molecular electronics sensor, when interpreting the single stranded DNA oligomer.

45. The system of claim 44, wherein the set of information is binary.

46. The system of claim 44, further comprising at least one of error detecting schemes or error correction schemes for minimizing errors within the single stranded DNA oligomer.

47. The system of claim 46, wherein the error detecting schemes are selected from repetition code, parity bits, checksums, cyclic redundancy checks, cryptographic hash functions and hamming codes, and the error correction schemes are selected from automatic repeat request, convolutional codes, block codes, hybrid automatic repeat request and Reed-Solomon codes.

48. The system 44, wherein the device comprises a CMOS chip based array of actuator pixels for DNA synthesis, the actuator pixels directing voltage/current or light-mediated deprotection within an enzymatic DNA synthesis reaction comprising phosphoramidite chemistries.
